(19) United States
(12) Plant Patent Application Publication
(10) Pub. No.: US 2003/0024018 P1
(43) Pub. Date: Jan. 30, 2003

(54) Floribunda rose plant named "KRA BENTIG'

(76) Inventor: Timm R. Kralovetz, Plano, TX (US)

Correspondence Address: CHRISTIE, PARKER & HALE, LLP, 350 WEST COLORADO BOULEVARD, SUITE 500, PASADENA, CA, 91105, US

(21) Appl. No.: 09/917,110

(22) Filed: Jul. 27, 2001

(51) Int. Cl. A01H 5/00

(52) U.S. Cl. PLT/143

(57) ABSTRACT

A floribunda rose plant producing attractive pink, orange, yellow bi-color flowers and a vigorous compact plant with dark green and burgundy foliage.

### BACKGROUND OF THE INVENTION

The present invention relates to a new and distinct floribunda rose variety which was originated by my crossing as seed parent the variety known as ‘PLAYBOY’ and as pollen parent the variety known as ‘FRAGRANT CLOUD’. The varietal denomination of this new rose is ‘KRA BENTIG’.

### SUMMARY OF THE INVENTION

Among the novel characteristics possessed by the new variety which distinguish it from its parents and all other varieties of which I am aware are its attractive, bi-color flowers.

Asexual reproduction by vegetative cuttings of the new variety as performed in Plano, Texas, shows that the foregoing and other distinguishing characteristics come true to form and are established and transmitted through succeeding propagation. Other known forms of asexual reproduction for roses, may be also employed.

### BRIEF DESCRIPTION OF ILLUSTRATIONS

The accompanying photographic illustrations show typical specimens of the vegetative growth and flowers of the new variety in different stages of development and depicted in color as nearly true as it is reasonably possible to make the same in a color illustration of this character.

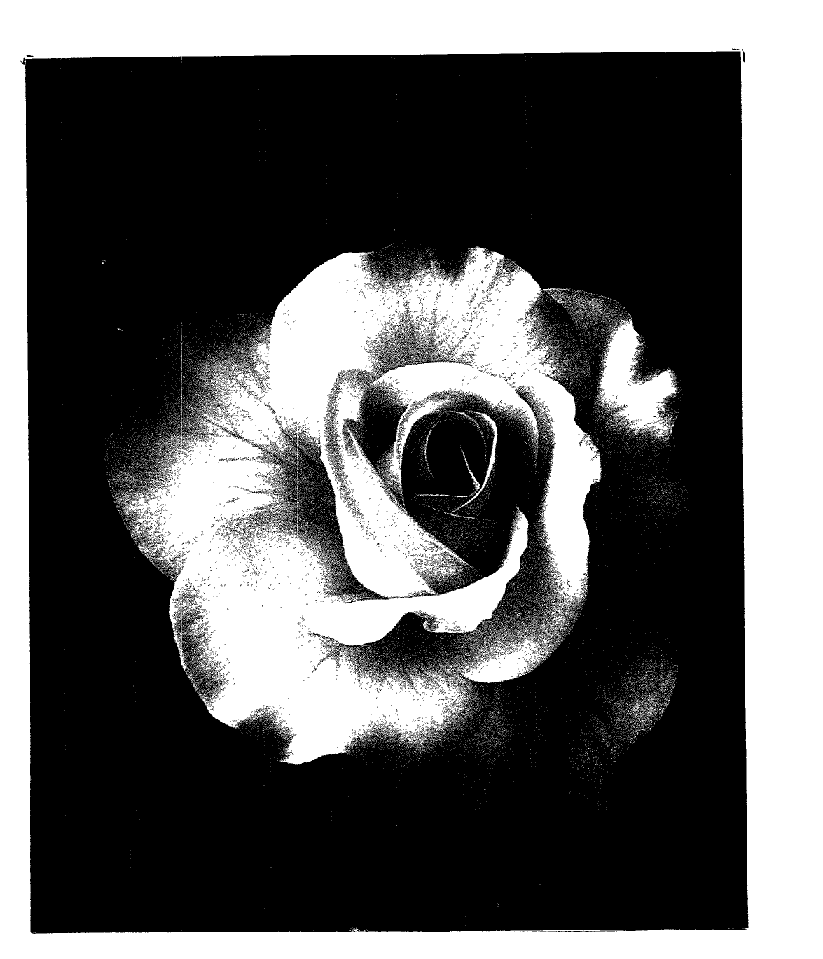

FIG. 1 shows a flower of the new variety; and

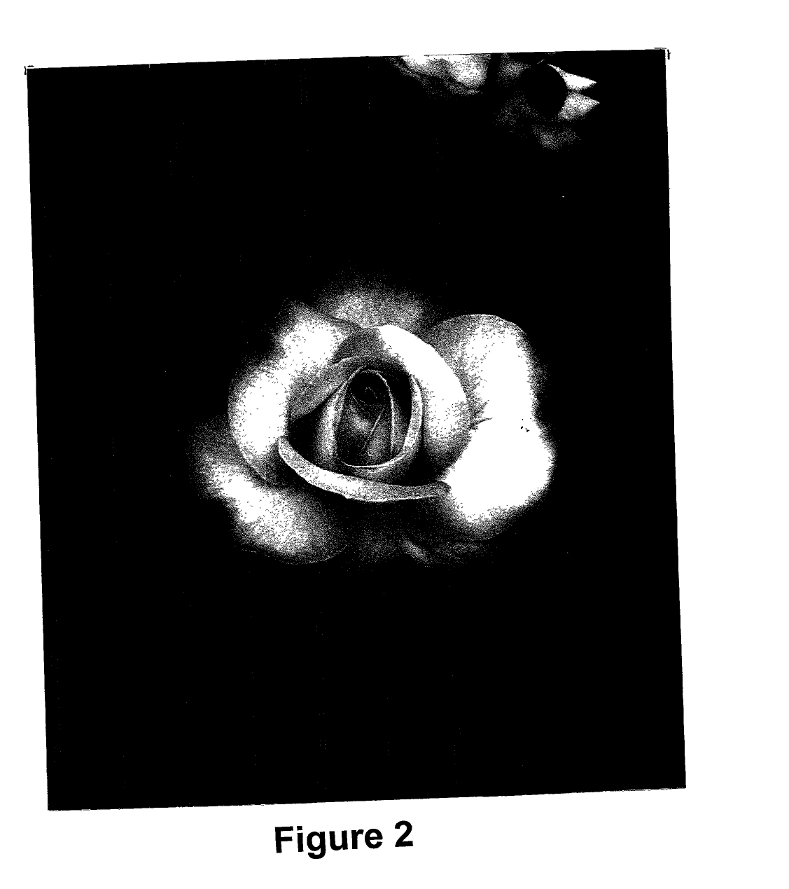

FIG. 2 shows flower and foliage of the new variety.

### DESCRIPTION OF THE NEW VARIETY

Parentage: Seedling

Seed parent.—‘PLAYBOY’

Pollen parent.—‘FRAGRANT CLOUD’

Class: Floribunda

The following is a detailed description of the new variety, from specimens grown outdoors in Plano, Tex. The color terminology used is in accordance with The Royal Horticultural Society Colour Chart (RHSCC), and refers to plate numbers in the aforementioned color chart. Phenotypic expression may vary depending on light, environmental and cultural conditions.

### FLOWER

Blooming Habit: Continuous

Bud

Size.—Large, about 1 inch.

Form.—Pointed.

Color.—Green bud near 138B.

Sepals.—Long, some sepals foliaceous, sepals cover bud, then divide and fold back at maturity; about 3 to 4 cm in length.

Color.—Near 74B to 74C at upper portion to near 11C to 11D toward base.

Peduncle

Length.—About 9 cm to about 12 cm.

Aspect.—Rough

Strength.—Erect, sturdy Color: Near 187B to lighter shades.

Bracts.—Near 137A.

Bloom

Size.—Average size when fully expanded—about 5 ½ inches.

Borne.—Singly.

Form.—High centered, exhibition type at first; petals imbricated, outer petals.

Petalage.—Number of petals under normal conditions—about 30 to 36

Color (⅓ to ½ open)

Top surface.—Outer picate edge near 69D washing into near 76D; lower portion of petal near 19B to 19C.

Under surface.—Outer edge near 69C washing into near 19C.

Center.—Color where petals bunch near 38A.

General tonality from a distance: Attractive plant, bi-color mango, yellow

Petals

Texture.—Medium thick.

Appearance.—Inside slightly matte and outside matte.

Form.—Orbicular, fan-shaped.

Arrangement.—Imbricated.

Petaloids.—Few.

Persistence.—Petals drop off cleanly.

Fragrance.—Light to moderate.

### PLANT

Form: Small to compact, growing to about 4 ft by 4 ft bush

Growth: Vigorous

Foliage: Compound, 3 to 5 leaflets

Size.—Medium.

Quantity.—Abundant.

Color.—New foliage: Near 138A to 138B Mature foliage: near 189A with Burgundy cast expressly at some edges, underside similar coloring.

Shape.—Ovate

Texture.—Upper side: leathery, semi-glossy Under side: leathery.

Edge.—Serrated.

Serration.—Serrate.

Leaf stem.—Color—green with burgundy cast.

Leaf apex.—Acute.

Leaf base.—Obtuse.

Resistance to disease: Very resistant to mildew, rust and blackspot

### WOOD

Color: Near 187A to 187D tinged with green bark smooth

Flower stem: About 4 to 11 inches from last cut

Thorns

Quantity.—Few, near 185A

Winter Hardiness: Has survived 24° F. to 106° F.

I claim:

1. A new and distinct floribunda rose plant, substantially as shown and described.
